(19) United States
(12) Patent Application Publication
(10) Pub. No.: US 2026/0283645 A1
(43) Pub. Date: Sep. 24, 2026

(54) Airway Implant System and Method

(71) Applicant: Somnus Technologies, Inc., GREENVILLE, WI (US)

(72) Inventors: Johnathan Howard Cronin, Greenville, WI (US); William Matthew Cronin, Greenville, WI (US)

(21) Appl. No.: 19/228,711

(22) Filed: Jun. 4, 2025

Related U.S. Application Data
(60) Provisional application No. 63/726,242, filed on Nov. 28, 2024.

(51) Int. Cl. A61B 17/34; A61B 17/00; A61B 17/24; A61B 90/00

(52) U.S. Cl. CPC A61B 17/3468; A61B 2017/00424; A61B 2017/248; A61B 2090/062

(57) ABSTRACT

A palatal implant includes a longitudinal biocompatible fibrous body having a body surface, a first end, and a second end, the second end including an anti-backout portion. Another palatal implant includes a longitudinal biocompatible fibrous body having a body surface, a first end, and a second end, the first end including a tapered portion. An implant inserter and system, and a method for delivering an implant into a soft palate of a patient, are also disclosed.

### FIELD

The present invention generally relates to an airway implant, system and method of treating snoring and obstructive sleep apnea. More particularly, the present invention relates to a method, system and device for a minimally invasive palatal implant, inserter and cartridge system and method of use.

### BACKGROUND

The present invention is directed to an apparatus, and system designed to treat airway conditions in patients. Such airway conditions may contribute to snoring and obstructive sleep apnea which can lead to disruptive sleep and related health issues. Data shows that snoring affects approximately 40% of adult men and 24% of adult women. Obstructive sleep apnea is estimated to affect 1 billion people worldwide, with 425 million suffering from moderate to severe sleep apnea. The prevalence in diagnoses of sleep disorders is increasing with the rate of comorbidities such as rising incidences of obesity, and growing awareness about the health consequences of untreated sleep disorders.

Currently available treatment options include many challenges: low patient compliance with some treatments, especially CPAP and higher cost, more aggressive, invasive procedures. Traditional methods for deploying airway implants often face challenges in precision and safety. The novel system disclosed herein may include a cartridge for use in conjunction with an applicator and handle, enabling the operator to deploy implants easily and reliably. The kit encompasses multiple implants within each cartridge, alongside the applicator, offering medical practitioners a comprehensive tool for airway treatment.

The airway implant and inserter system, along with the cartridge kit, represents a significant advancement in the treatment of airway conditions. By integrating a user-friendly applicator with precise implant deployment capabilities, the present invention addresses common deficiencies in the prior art, such as: implant placement, precision, safety, and ease of use. The ergonomic design and efficient mechanism aim to improve both clinical outcomes for patients and operational efficiency for healthcare providers. The minimally invasive nature of the present system and method address the needs of patients seeking alternatives to CPAP or more invasive surgeries. There is a need for a system that ensures efficient and minimally invasive implant delivery, providing an innovative solution for airway treatment.

The procedure according to an embodiment of the present invention, improves airway patency by reinforcing the soft palate with submucosal implants, reducing its tendency to collapse or vibrate during sleep. The design of the system according to an embodiment of the present invention ensures:
Accurate depth-controlled placement
Repeatable deployment
Minimal trauma to surrounding tissues

### SUMMARY

The present invention presents a sophisticated apparatus, systema and method for treating airway conditions, utilizing a novel implant deployment system. The device, systems and method of the present invention are directed to an unmet need in efficiently treating airway and sleep disorders. The system includes an inserter tool includes depth control and an insertion trial for seamless, targeted implant deployment. The system further includes a hollow needle that is pre-loaded with an airway implant, and includes a trocar-shaped distal tip, ensuring precise tissue penetration and implant delivery. The sterile kit, housing multiple pre-loaded implant insertion needles, allows for sequential, controlled release of each implant, optimizing the treatment outcome. This invention simplifies the deployment process, increasing treatment reliability and safety, thereby marking a significant advancement in airway management.

The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DESCRIPTION OF THE DRAWINGS

FIG. 1 depicts a perspective view of a delivery system according to an embodiment of the present invention

FIG. 2 depicts an implant of the present invention loaded into a needle of the present invention

FIG. 2a depicts an embodiment of an implant according to the present invention

FIG. 3 depicts an implant partially delivered out of a needle if the present invention

FIG. 4 depicts a delivery system kit according to an embodiment of the present invention

FIG. 5 depicts another embodiment of a delivery system kit according to the present invention

FIG. 6 depicts yet another embodiment of a delivery system according to the present invention

FIG. 7 depicts an alternate embodiment of the present invention

FIG. 8 depicts a trial guide according to an embodiment of the present invention

FIG. 9 depicts a guide holder according to an embodiment of the present invention

FIG. 10 depicts an alternate embodiment of a guide holder according to an aspect of the present invention

FIG. 11 depicts an awl according to an embodiment of the present invention

FIG. 12 depicts a punch awl according to an aspect of the present invention

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the spirit and scope of the invention as defined by the appended claims.

### DETAILED DESCRIPTION

In the following descriptions, the present invention will be explained with reference to example embodiments thereof, including a surgical method and tools used to perform the method. However, these embodiments are not intended to limit the present invention to any specific example, embodiment, environment, applications or particular implementations described in these embodiments. Therefore, description of these embodiments is only for illustration, rather than to limit the present invention.

It should be appreciated that dimensional relationships among individual elements in the attached drawings are illustrated only for ease of understanding, but not to limit the actual scale.

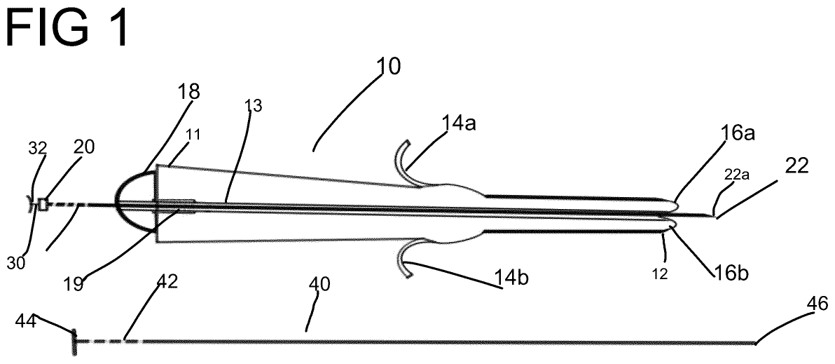

In an embodiment of the present invention, as is shown in FIG. 1, the total delivery system (TDS) may include an implant delivery tool 10. Tool 10 may include a proximal end 11 and a distal end 12. Tool 10 may further include a cannulation 13 running therethrough from proximal end 11 through distal end 12. According to an aspect of the present invention, tool 10 may include finger grips 14a and 14b. In an embodiment, tool 10 may include an adjustable depth stop 18 at proximal end 11. In one embodiment depth stop 18, may be a silicon palm stop configured to engage a user's palm in use. Depth stop 18 may be operably engaged to a depth adjustment mechanism 19 seated in proximal end 11. In an embodiment, depth stop 18 may include a threaded male portion that mates with depth adjustment 19. In such an embodiment, depth adjustment 19 may be a threaded female portion. In an embodiment, stop 18 may be rotated 360 degrees about the threaded engagement for each one-millimeter movement. In another embodiment stop 18 and mechanism 19 may comprise a pop-lock movement wherein each click equates to 5 millimeters of movement. Other depth adjustment mechanisms known in the art are contemplated and may be used.

Tool 10 may further include bumpers 16a and 16b incorporated into or onto distal end 12. Bumpers 16a and 16b may be comprised of silicone or any other biocompatible material that provides cushion and/or pliability.

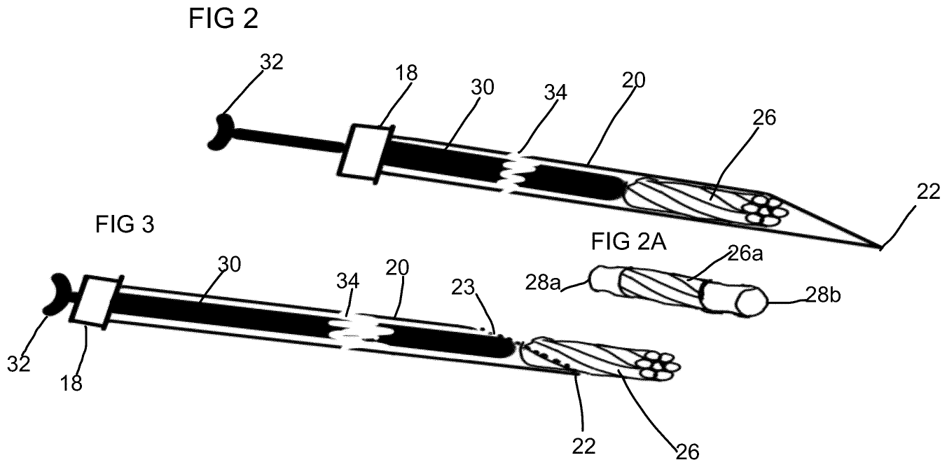

According to an embodiment, tool 10 is configured to receive implant loaded needle 20 through cannulation 13. Needle 20 may be axially cannulated such that it may be pre-loaded with implant 26, as is shown in FIGS. 2 and 3. In an embodiment needle 20 may include a trocar tip 22, or other sharp edge configured to create an implant insertion path through soft tissue. Needle 20 enables precise penetration with minimal trauma, featuring axial cannulation for implant passage through needle bore 22a. In an aspect, needle 20 may include an anodized distal tip. Needle 20 may further include depth markings 24. Depth markings 24 may be spaced apart at 5 mm intervals, although other intervals are contemplated. In an embodiment, depth markings 24 may measure up to a 20 mm measure. According to an aspect of the present invention, depth markings 24 are timed to depth stop 18.

As can be seen in FIG. 1, the delivery system of the present invention may include trial 40. In an embodiment trial 40 may include push bar 44 and depth markings 42. Depth markings 42 match depth markings 24 and are timed to depth stop 18 enabling the user to safely determine the desired insertion depth with trial 40, prior to placement of needle 20. Trial 40 may further include a blunt distal tip 46 to prevent patient injury. In an aspect push rod 30 may be used to advance implant 26 out of needle 20. Push rod 30 may include thumb rest 32, or other suitable user engagement mechanism.

In an embodiment of the present invention, needle 20 may be axially cannulated such that implant 26 may be loaded into needle 20 during the manufacturing process. In such an embodiment, needle 20 is pre-loaded with implant 26 prior to needle 20 being placed through tool 10. FIG. 2 shows a perspective view, including scale break 34, of needle 20 having implant 26 fully seated therewithin. As is shown in FIG. 3, including scale break 34, in an embodiment push rod 30 may operably engage with implant 26, advancing implant 26 out of needle 20 and into soft tissue.

Implant 26 may be sized and configured for placement in a patient's airway tissue such that implant 26 provides structural and therapeutic functions. Airway implant 26 may be constructed from any biocompatible material. In one aspect, airway implant 26 may be comprised of polyethylene terephthalate (PET), or any other suitable polyester. In another embodiment, implant 26 may be comprised of any polymer in the PETG category, including but not limited to ultra-high molecular weight polyethylene (UHMWPE). Airway implant 26 may be braided, knitted, twisted, form molded, extruded or otherwise constructed of any biocompatible material and/or combination of biocompatible materials, including resorbable, non-resorbable and/or a combination thereof. In an embodiment, implant 26 may be braided, knitted or otherwise constructed with different braiding or knitting densities. In another aspect, implant 26 may be comprised of synthetic or biologic material and/or a combination thereof. In an embodiment, implant 26 may be surface coated or embedded with a material to induce or encourage fibrous tissue growth.

In another embodiment, implant 26 may include a roughened surface, mechanical, chemical and or thermal texturing, to increase surface area and encourage tissue growth.

In one aspect, implant 26 may be sized and configured to accommodate different airway anatomies, enabling tailored solutions for specific populations. In an embodiment, implant 26 may be tapered at its leading edge. Implant 26 may include crimped ends 28a and 28b, as is shown in FIG. 2a. Crimped ends may be comprised of nitinol, stainless steel or any other desired biocompatible material. Implant 26 may further include structural supports to stiffen implant fibers enabling easier insertion. In an embodiment, implant 26 may include biomedical markers, such as for example tantalum. In one aspect a leading edge of implant 26 may be capped with any type of bio material. In an embodiment, implant ends 28a and 28b may be manufactured with a conditioned clean cut or an end blob. Implant ends 28a and 28b may further be configured with an anti-backout feature, such as for example a T-shape, crimped end, blob or bulbous end portion, and/or other feature.

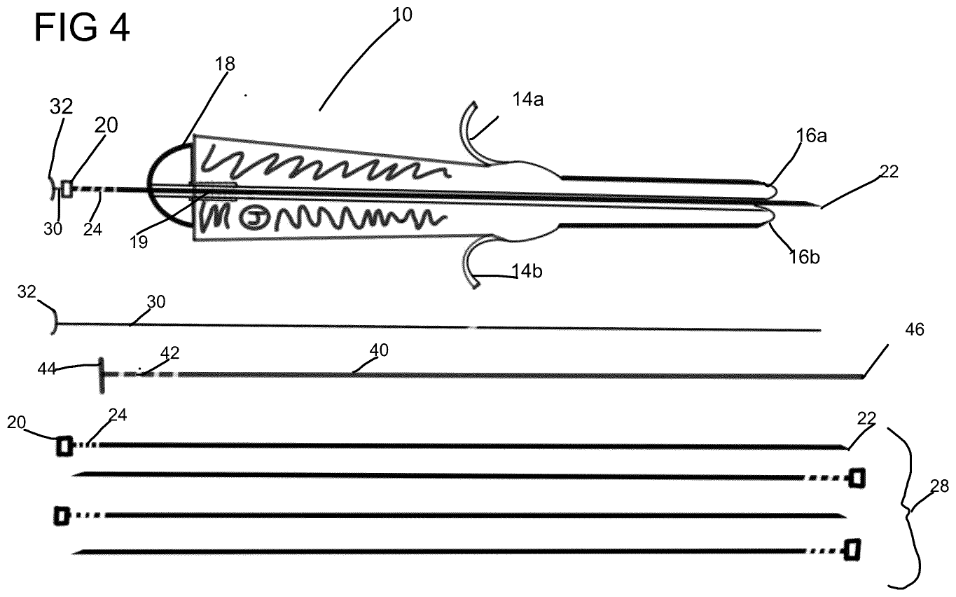

According to one aspect of the present invention, as is shown in FIG. 4, the present invention may include a delivery system including a sterile packed kit. In such an embodiment, the kit may include delivery system 10, trial 40 and a plurality of implant preloaded needles 28. In a preferred embodiment, a kit according to the present invention may include in the range of 3 to 5 pre-loaded needles 20.

In an embodiment of the present invention, a method of using delivery system 10 may include administering a local anesthetic to a patient. Said patient may then be positioned in a reclined supine position with their mouth open to provide access to the oral cavity. According to one aspect, a user may identify depth stop 18 at proximal end 11 of total delivery system 10. Depth stop 18 may be rotated counterclockwise to expose first, second and third depth markings 24. Depth stop 18 may be rotated until user feels a tactile stop. In an embodiment, maximum depth stop may be 20 mm, although other lengths are contemplated. Depth stop may be backed out to its maximum length. Once the depth is secured, bumpers 16a and 16b may be positioned just distal to the hard palate, docking delivery system 10 onto a soft palate of a patient in a midline fashion.

In an embodiment, once delivery system 10 is safely docked, trial 40 may be inserted through a cannulation in depth stop 18 at proximal end 11 of delivery system 10. Verify that the proximal end of trial 40 meets depth stop 18, ensuring maximum protection, and that trial 40 is properly aligned and in position. Desired alignment may be confirmed using X-ray imaging.

This ensures proper depth and positioning in preparation for implant 26.

According to an aspect of the present invention, a user may determine the desired implant 26 depth and adjust the depth stop 18 clockwise to align the appropriate mark 24 with the proximal end of trial 40. For example, for a 20 mm depth, rotate to the fourth mark 24; for 15 mm, rotate to the third mark 24. Once the correct depth is confirmed, trial 40 may be removed and implant delivery needle 20 may be inserted through a cannulation in depth stop 18 into delivery system 10. Needle 20 may be advanced until a tactile stop is felt, ensuring the proximal end of needle 20 is flush with the depth stop 18. Push rod 30 may then be advanced using thumb rest 32 through delivery needle 20 until another tactile stop is felt, securing implant 26 firmly in place.

In an embodiment a user may confirm that implant 26 is properly positioned for optimal airway function. Implant 26 placement may be adjusted as needed for patient comfort and efficacy. Once satisfied with placement of implant 26, opposing force vectors may be applied through thumb rest 32 to secure implant 26, while retracting finger positioners 16a and 16b to remove the TDS system 10, leaving implant 26, needle 20, and push rod 30 in position. In an embodiment, both needle 20 and push rod 30 may then carefully be removed.

According to an aspect, a second implant 26 may then be inserted by shifting TDS 10 approximately in the range of 5 mm off the midline in the sagittal plane, depending on the specific patient's anatomy. Bumpers 16a and 16 may be adjusted and docked for proper alignment of a second implant 26 as in the implantation process described hereinabove for the first implant 26. The implantation process may be repeated as necessary for the desired number of implants 26, ensuring accurate depth and positioning with each trial 40 and needle 20 insertion. This ultra-minimally invasive approach efficiently treats snoring and mild to moderate obstructive sleep apnea by precisely positioning implants in the soft palate, enhancing airway stability.

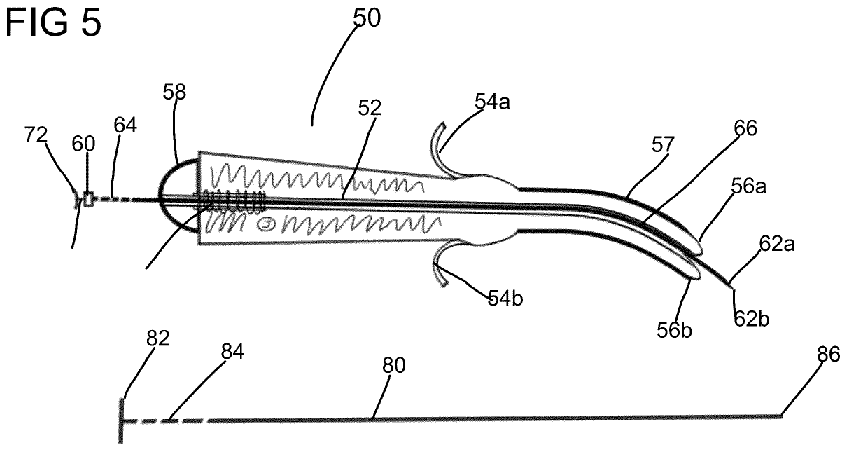

FIG. 5 depicts another embodiment of the present invention. According to one aspect, insertion delivery tool 50. Tool 50 may include a proximal end 51 and a distal end 52. Tool 50 may further include a cannulation 53 running therethrough from proximal end 51 through distal end 52. According to an aspect of the present invention, tool 50 may include finger grips 54a and 54b. In an embodiment, tool 50 may include a curved portion 57 to mimic a patient's anatomy. In one embodiment, tool 50 may be comprised of flexible material to conform to a patient's anatomy upon insertion into said patient's tissue.

In an embodiment, tool 50 may include an adjustable depth stop 58 at proximal end 51. In one embodiment depth stop 58, may be a silicon palm stop configured to engage a user's palm in use. Depth stop 58 may be operably engaged to a depth adjustment mechanism 59 seated in proximal end 51. In an embodiment, depth stop 58 may include a threaded male portion that mates with depth adjustment 59. In such an embodiment, depth adjustment 59 may be a threaded female portion. Other depth adjustment mechanisms known in the art are contemplated and may be used.

Tool 50 may further include bumpers 56a and 56b incorporated into or onto distal end 52. Bumpers 56a and 56b may be comprised of silicone or any other biocompatible material that provides cushion and/or pliability.

According to an embodiment, tool 50 is configured to receive implant loaded needle 60 through cannulation 53. Needle 60 may be axially cannulated such that it may be pre-loaded with implant 66, as is shown in FIGS. 2 and 3. In an embodiment needle 60 may include a trocar tip 62, or other sharp edge configured to create an implant insertion path through soft tissue. Needle 60 enables precise penetration with minimal trauma, featuring axial cannulation for implant passage through needle bore 62a. Needle 60 may further include depth markings 64. According to an aspect of the present invention, depth markings 64 are timed to depth stop 58. According to one aspect, needle 60 may include a curved portion 60 to mimic a patient's anatomy. In one embodiment, needle 60 may be comprised of flexible material to conform to a patient's anatomy upon insertion into said patient's tissue.

As can be seen in FIG. 5, the delivery system of the present invention may include trial 80. In an embodiment trial 80 may include push bar 84 and depth markings 82. Depth markings 82 match depth markings 64 and are timed to depth stop 58 enabling the user to safely determine the desired insertion depth with trial 80, prior to placement of needle 60. Trial 80 may further include a blunt distal tip 86 to prevent patient injury. In an embodiment, trial 80 may be comprised of flexible material to conform to a patient's anatomy upon insertion into said patient's tissue. In an aspect push rod 70 may be used to advance implant 66 out of needle 60. Push rod 70 may include thumb rest 72, or other suitable user engagement mechanism.

According to one aspect of the present invention, delivery system 50 may be kitted into a sterile packed kit which may include delivery system 50, trial 80 and a plurality of curved pre-loaded needles 60.

The method of using delivery system 50 includes the same process as is described hereinabove for delivery system 10. Delivery system 50 further includes curved portion 57 and corresponding curved needle portion 66.

When choosing between a straight instrument versus a curved instrument for the Total Delivery System (TDS) used in airway implant procedures, several factors come into play, primarily related to ergonomics and surgeon preference. The benefits of a straight instrument may include more precision and control because a straight instrument's linear shape often provides the user better alignment during insertion and manipulation. Further the simplicity of design for a straight instrument can be more efficient to manufacture and maintain, reducing the risk of mechanical issues during procedures. Users accustomed to straight instruments may also find them easier to handle, especially if they have been trained primarily with straight tools.

Curved Instruments also feature user benefits, including ergonomic comfort. A curved instrument can offer enhanced ergonomic comfort, especially when accessing hard-to-reach areas. The curve may align better than a straight instrument, with the natural contours of the oral cavity and soft palate. In addition, curved instruments might provide better access to the distal airway and soft tissue areas, allowing for more effective navigation around anatomical structures. Further, ergonomically designed curved instruments can reduce user fatigue during lengthy procedures by allowing a more natural hand position.

Ultimately, the choice between straight and curved instruments often comes down to the user's personal preference based on their experience, training, and comfort level with the instruments. Certain cases may benefit from the unique advantages of one type over the other, depending on the specific anatomy and procedural goals. Both instrument types have their benefits, and the decision often reflects a combination of ergonomic considerations, patient anatomy and personal preferences of the user team.

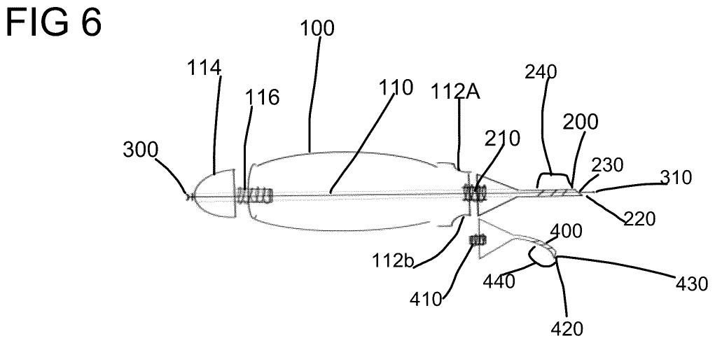

FIG. 6 depicts an alternative embodiment of the present invention. In this embodiment, total delivery system 100 may include cannulation 110 along its axis. System 100 may further include finger grips 112a and 112b at its distal end. In an embodiment system 100 may include depth stop 114 at its proximal end. Depth adjustment mechanism 116 may be operably engaged in the proximal end of system 100 and configured to mate with depth stop 114. In an embodiment of the invention depth stop 114 may include male threads to operable engage with female threads on depth adjustment mechanism 116.

In one aspect of the invention, system 100 may include a hollow needle 200 preloaded with an airway implant. Said airway implant may be of the type described hereinabove in system 10. As is shown in FIG. 6, needle 200 may include a straight shaft, a trocar tip 220, a bore opening 230 and depth markings 440. Needle 200 may further include an engagement mechanism 210 to couple the proximal end of needle 200 to the distal end of system 100. In an embodiment, mechanism 210 may be a threaded screw coupling, a pinch-lock mechanism, or any other suitable coupling mechanism.

In another aspect of the invention, system 100 may include a hollow needle 400 preloaded with an airway implant. Said airway implant may be of the type described hereinabove in system 10. As is shown in FIG. 6, needle 400 may include a curved shaft, a trocar tip 420, a bore opening 430 and depth markings 440. Needle 400 may further include an engagement mechanism 410 to couple the proximal end of needle 400 to the distal end of system 100. In an embodiment, mechanism 410 may be a threaded screw coupling, a pinch-lock mechanism, or any other suitable coupling mechanism. System 100 may further include a push rod 300 having a blunt tip 310.

According to an embodiment of the present invention, TDS 100 may be packed in a sterile kit including TDS 100 and a plurality of pre-loaded implant delivery needles 200.

In an embodiment of the present invention, the method of using system 100 may include administering local anesthesia to the patient, and positioning the patient comfortably, in a reclined, supine position with their mouth open. A user may identify the depth stop 114 at the proximal end system 100. Depth stop 114 may be rotated counterclockwise to expose markings on a male threaded portion of depth stop 114, indicating 5 mm intervals. In an embodiment, markings may indicate a length up to 20 mm. According to an aspect of the present invention depth stop 118 may be backed out to a maximum of 20 mm mark in preparation of needle 200 attachment.

Pre-loaded implant needle 200 may be attached to the distal end of system 100 using mechanism 410. Push rod 300 may be inserted through the proximal end of depth stop 114, passing through the entire length of system 100 until a tactile hard stop is felt. Push rod 300 may be removed and set it aside. Desired implant depth may be determined by adjusting depth stop 114 accordingly, rotating to align with the appropriate depth mark. Once the correct depth is confirmed, system 100 may be placed within the oral cavity, targeting the midline of the soft tissue palate on a sagittal image dorsal to the hard palate.

In an embodiment an axial force may be applied through the depth stop 114 and TDS system 100, thereby creating a stab incision into the soft tissue palate and continue plunging to the desired depth marking 240 on the distal tip of needle 200. Proper positioning may be verified and pushrod 300 may be inserted through system 100 until a tactile stop to secure the implant at the predetermined depth. Implant may be secured using an axial force through TDS 100 by simultaneously removing/retracting system 100, leaving the implant in place.

According to an embodiment, one or more additional implants may be inserted. The first needle 200 may be removed from the TDS 100 by disengaging mechanism 210. In an embodiment wherein mechanism 210 is configured as a pinch-lock, a pinch-and-hold grip may be performed, rotating needle 200 counterclockwise 90 degrees, disengaging needle 200 form system 100. A second needle 200 may be locked into place with mechanism 210. In an embodiment wherein a pinch-lock is used as mechanism 210, a pinch-and-hold grip may be used, rotating 90 degrees in a clockwise fashion to secure needle 200 to system 100. The desired implant depth may be determined, and depth stop 114 adjusted accordingly, rotating to align with the desired depth mark. Once the correct depth is confirmed, system 100 may be placed approximately 5 mm off the midline of the soft tissue palate on a sagittal plane dorsal to the hard palate, or at a location specific to the patient's anatomy. The process then repeats as is described hereinabove for the first preloaded implant needle 200. Additional implants may be inserted following the same method and procedure to maximize optimal airway function for each individual anatomy.

In an embodiment, implant positioning for optimal airway function may be confirmed by x-ray, direct visual and tactile observation or other suitable measure. Any adjustments may be made and TDS 100 may be removed, leaving the implants in place. This method aims to enhance airway stability by strategically placing implants in the softer tissue palate, providing an effective treatment for mild to moderate Obstructive Sleep Apnea (OSA) and snoring.

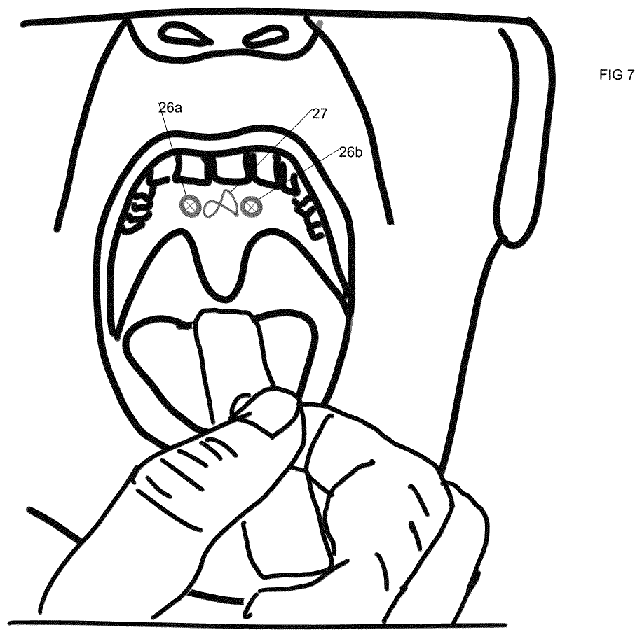

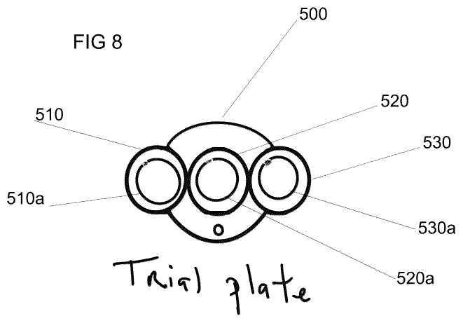

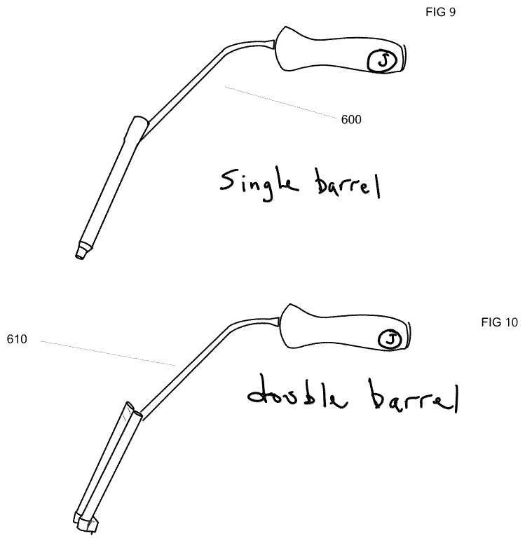

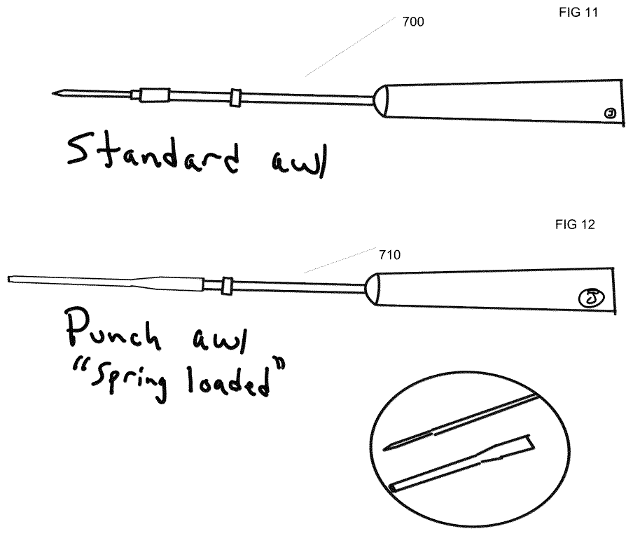

In another embodiment, implant 26 may be inserted from a mid-sagittal line perspective. A first implant 26a may be inserted into a one side of a patient's palate and a second implant 26b may be inserted into the opposite side of a patient's palate. As shown in FIG. 7, according to one aspect, a suture 27 may be placed between first implant 26a and second implant 26b, creating a trampoline effect.

In yet another embodiment, the method of inserting implant 26 may include the use of a single-use sterile kit that enhances precision and flexibility during implantation procedures. The kit may contain specialized instruments, as shown in FIG. 8, including a uniquely designed trial plate 500 constructed from a polymer, metal, polyetheretherketone (PEEK) or other suitable biocompatible material, equipped with integrated locking rings 510a, 520a, 530a, each hole 510, 520, 530. A kit according to an embodiment may further include trial plate guide holders 600, 610. Holders 600,610 may be configured as a single 600 and/or double-barreled 610 guide. According to one aspect a double-barrel guide 610 may attach securely into locking rings 510a and 530a of trial plate 500, providing clinicians the capability to adjust implant 26 trajectories up to the range of about 30 degrees medially, laterally, cephalad, and caudad at the two outermost holes 510, 530. The central hole 520 on trial plate 500 offers minimal variability, acting as a fixed reference point. In an embodiment, an inserter, using a configuration such as for example double-barreled guide 610, may be configured to insert more than one implant 26 at the same time.

In an embodiment, clinicians may utilize implant insertion instruments such as for example, a straight inserter 700 or a spring-loaded punch awl inserter 710 may be preloaded with implant 26 through guides 600, 610. In certain embodiments, a user may first select a guide holder 600, 610—either fixed-angle or variable-angle—depending on their desired level of control and procedural workflow. The fixed-angle guide holders may feature built-in, biased trajectories that converge at predetermined angles. These holders may be designed to streamline the procedure by eliminating the need for manual angle adjustment, supporting consistent implant positioning, reduced procedure time, and optimized workflow. Both insertion instruments and implants are passed through the fixed guide holder, but the clinician cannot alter the trajectory.

Alternatively, variable-angle guide holders may allow the healthcare provider to set custom trajectories at the time of the procedure. This option offers full control over angle and direction, enabling greater adaptability based on patient-specific anatomy or clinical judgment. As with the fixed holders, instruments and implants pass through the guide, but the path is determined entirely by the user without any built-in bias.

In another embodiment, implant insertion instruments may be disposable. In yet another embodiment, insertion instruments may include a tapered distal aspect, providing healthcare providers with both variable and fixed options without the complexity and additional cost associated with snap rings. This technique disclosed herein provides a robust system characterized by built-in safety mechanisms, reproducible outcomes, and enhanced flexibility, empowering healthcare providers to achieve consistent, safe, and effective treatment results for patients suffering from snoring and mild to moderate OSA.

In an embodiment, method for delivering a submucosal implant into a soft palate of a patient may include using a Total Delivery System (TDS). In one aspect, a TDS may include a handle with an adjustable palm stop, a distal targeting needle adaptor preloaded with an implant, and a pushrod with a proximal thumb stop. A palm stop may be rotated to expose depth markings and adjusted to a predetermined depth setting. A distal targeting needle adaptor may be attached to the TDS handle using a locking mechanism.

A pushrod may be inserted through the TDS system to confirm an unobstructed passage. Once confirmed, the pushrod may be removed. According to an aspect, the TDS system may then be placed intraorally against the patient's soft palate and axial pressure may be applied to puncture the tissue and advance the TDS to the preset depth. The pushrod may then be re-inserted to deliver the implant to the target site. In an embodiment,
the TDS system may be simultaneously retracted while applying axial force through the pushrod, thereby deploying and securing the implant in place within the soft palate.

According to an embodiment, insertion steps may be repeated with additional implants as desired. In such an embodiment, each subsequent implant may be delivered lateral to the midline of the soft palate at clinically desired distances. confirming final implant positioning may be confirmed for airway stabilization and any adjustments in placement may be made if necessary.

A system according to an embodiment of the present invention may include preparing the system by setting up the system. In an embodiment system set up may include, a surgical technologist or dental assistant opening and preparing a sterile-packed TDS kit. According to one aspect a TDS kit may include, at least: a TDS handle with integrated depth-adjustable palm stop, three distal needle adaptors pre-loaded with implants and one pushrod with a proximal thumb stop. Once the TDS kit is set up, local anesthesia may be administered to the soft palate region and the patient may be positioned supine, with their mouth open while maintaining a sterile field.

In an embodiment of the present invention, the palm stop may be rotated counterclockwise to expose depth markings in the range of about 5 mm increments (up to about 20 mm). The palm stop may be backed out fully to the 20 mm mark. A distal targeting needle adaptor (preloaded with implant) may be attached to the distal end of the TDS handle using the pinch-lock engagement mechanism. According to an aspect, the pushrod may be inserted from the proximal end through the TDS system until a tactile hard stop is reached, confirming clear passage. The pushrod may then be removed.

A first implant insertion according to an embodiment of the invention may include the steps of depth adjustment by rotating the palm stop to the desired implant depth (e.g., 15 mm) using the visual indicators on the handle. In an embodiment, the palate may be accessed by introducing the TDS system intraorally, targeting the midline of the soft palate, dorsal to the hard palate. Axial pressure may be applied through the palm stop to puncture the soft palate, creating a linear insertion path down to the preset depth mark on the needle adaptor. In an aspect, the implant may be deployed by reinserting the pushrod through the TDS 2.0 system until a firm stop is felt, confirming the implant is seated at the target depth. Axial force may continue to be applied through the handle while simultaneously retracting the TDS system, leaving the implant securely embedded in the tissue.

Additional implants may be placed as desired, in an embodiment, such additional implants may be placed in lateral insertion sited by removing the used needle adaptor by pinch-holding and rotating counterclockwise 90°, A second distal needle adaptor may be attached using a pinch-and-rotate clockwise motion. A desired implantation depth may be set on the palm stop. In an embodiment, the TDS system may be positioned in the range of about 5 mm lateral to the midline, dorsal to the hard palate. Palatal puncture, pushrod implant deployment and TDS system retraction may be repeated as with the first implant insertion.

In an embodiment, a third implant may be desired. In such an embodiment, the second needle adaptor may be removed and a third needle adaptor may be attached. The desired depth may be adjusted using the pam stop as with the first two implants. The user may target the contralateral 5 mm lateral position from midline. The implant may be deployed using the same insertion and retraction technique as with the first two implants. Additional implants may be inserted by repeating the same steps as is described herein above.

According to an aspect, a user may wish to confirm implant placement. This may be accomplished via tactile feedback or imaging (if applicable). A user may ensure all implants are fully seated within the tissue and do not exhibit signs of extrusion or misplacement. Any adjustments or repeat procedure may be made as clinically indicated.

Embodiments of the present invention may include disposable and/or reusable components, balancing cost-effectiveness and sustainability. Variants of the present invention may be developed for various settings, enhancing versatility from emergency to surgical use.

The present invention may be embodied in other specific forms without departing from the spirit or essential attributes thereof, and it is, therefore, desired that the present embodiment be considered in all respects as illustrative and not restrictive. Those skilled in the art may recognize other equivalents to the specific embodiment described herein which equivalents are intended to be encompassed by the claims attached.

1. A palatal implant comprising:
a longitudinal biocompatible fibrous body having a body surface, a first end, and a second end, the second end including an anti-backout portion.

2. The implant of claim 1, wherein the biocompatible fibrous body is comprised of ultra-high molecular weight polyethylene.

3. The implant of claim 1, wherein the biocompatible fibrous body is comprised of a polyester.

4. The implant of claim 1, wherein the fibrous body surface includes surface texturing.

5. The implant of claim 1, wherein the first end and the second end include biological markers.

6. The implant of claim 1, wherein the second end includes a bullet nose.

7. The implant of claim 6, wherein the bullet nose is crimped.

8. A palatal implant comprising:
a longitudinal biocompatible fibrous body having a body surface, a first end, and a second end, the first end including a tapered portion.

9. The implant of claim 8, wherein the biocompatible fibrous body is comprised of ultra-high molecular weight polyethylene.

10. The implant of claim 8, wherein the biocompatible fibrous body is comprised of a polyester.

11. The implant of claim 8, wherein the fibrous body surface includes surface texturing.

12. The implant of claim 8, wherein the first end and the second end include biological markers.

13. The implant of claim 8, wherein the second end includes a bullet nose.

14. The implant of claim 13, wherein the bullet nose is crimped.

15. An implant inserter comprising:
a handle with an adjustable palm stop;
a distal targeting needle adaptor preloaded with an implant; and
a pushrod with a proximal thumb stop.

16. The inserter of claim 15, wherein the inserter further includes depth markings.

17. The inserter of claim 15, wherein the inserter further includes a locking mechanism.

18. A system comprising:
at least one palatal implant; and
at least one palatal implant inserter sterile packed for single use.

19. A method for delivering an implant into a soft palate of a patient comprising:
providing an inserter comprising a handle with an adjustable palm stop, a distal targeting needle adaptor preloaded with an implant, and a pushrod with a proximal thumb stop;
rotating the palm stop to expose depth markings and adjusting to a predetermined depth setting;
attaching the distal targeting needle adaptor to the handle using a locking mechanism;
inserting the pushrod through the inserter to confirm unobstructed passage and removing the pushrod;
placing the inserter intraorally against the soft palate of the patient and applying axial pressure to puncture tissue of the soft palate and advance to the preset depth;
re-inserting the pushrod to deliver the implant to a target site in the soft palate;
simultaneously retracting the inserter while applying axial force through the pushrod, thereby deploying and securing the implant in place within the soft palate;
repeating the providing, the rotating, the attaching, the inserting, the placing, the re-inserting, and the simultaneous retracting and applying for additional implants, wherein each subsequent implant is delivered lateral to a midline of the soft palate at defined distances; and
confirming final implant positioning for airway stabilization and making adjustments if necessary.
